(19) United States
(12) Patent Application Publication
(10) Pub. No.: US 2016/0287419 A1
(43) Pub. Date: Oct. 6, 2016

(54) IN-SITU FORMED STENT

(71) Applicant: BOSTON SCIENTIFIC SCIMED, INC., Maple Grove, MN (US)

(72) Inventors: MAN MINH NGUYEN, Harvard, MA (US); DONALD C. von LINDEN, JR., Lancaster, MA (US); ENDA P. McLAUGHLIN, Kinvara (IE); SEAN GRIFFIN, Galway (IE); MICHAEL WALSH, Galway (IE); THOMAS M. KEATING, Galway (IE); DAVID SHIEL, Galway (IE); SHANE DEVOY, Galway (IE)

(21) Appl. No.: 15/088,638

(22) Filed: Apr. 1, 2016

Related U.S. Application Data
(60) Provisional application No. 62/142,161, filed on Apr. 2, 2015.

(51) Int. Cl. A61F 2/95

(52) U.S. Cl. CPC A61F 2/95; A61F 2240/001; A61F 2210/0085

(57) ABSTRACT

A delivery system for forming a stent in-situ may comprise an elongate shaft having a proximal end and a distal end and a lumen extending from the proximal end towards the distal end of the elongate shaft. A plurality of apertures may be positioned adjacent to a distal end region of the elongate shaft. The plurality of apertures may extend from an outer surface to an inner surface of the elongate shaft and be in fluid communication with the lumen. A port may be affixed to the proximal end of the elongate shaft and in fluid communication with the lumen.

### CROSS-REFERENCE TO RELATED APPLICATIONS

This application claims priority under 35 U.S.C. §119 to U.S. Provisional Application Ser. No. 62/142,161, filed Apr. 2, 2015, the entirety of which is incorporated herein by reference.

### TECHNICAL FIELD

The present disclosure pertains to medical devices, and methods for manufacturing medical devices. More particularly, the present disclosure pertains to an in-situ formed stent.

### BACKGROUND

Implantable stents are devices that are placed in a body structure, such as a blood vessel or body cavity, to provide support and to maintain the structure open. These devices are manufactured by any one of a variety of different manufacturing methods and may be used according to any one of a variety of methods.

### SUMMARY

This disclosure provides design, material, manufacturing method, and use alternatives for medical devices. An example medical device may include a delivery system for forming a stent in-situ. The delivery system for forming a stent in-situ comprises:

an elongate shaft having a proximal end and a distal end;

a lumen extending from the proximal end towards the distal end of the elongate shaft;

a plurality of apertures adjacent a distal end region of the elongate shaft, the plurality of apertures extending from an outer surface to an inner surface of the elongate shaft and in fluid communication with the lumen; and

a port affixed to the proximal end of the elongate shaft and in fluid communication with the lumen.

Alternatively or additionally to any of the embodiments above, wherein the outer surface of the elongate shaft comprises a plurality of radially extending ridges and a plurality of valleys extending along a length of the elongate shaft.

Alternatively or additionally to any of the embodiments above, wherein the plurality apertures are disposed within at least one valley of the plurality of valleys.

Alternatively or additionally to any of the embodiments above, wherein the plurality of apertures comprises at least one aperture disposed in each valley of the plurality of valleys.

Alternatively or additionally to any of the embodiments above, wherein the plurality of apertures comprises a two or more apertures disposed in each valley of the plurality of valleys.

Alternatively or additionally to any of the embodiments above, wherein the two or more apertures are arranged in a linear array.

Alternatively or additionally to any of the embodiments above, wherein the outer surface of the elongate shaft comprises a radially extending helical ridge and a helical valley extending along a length of the elongate shaft.

Alternatively or additionally to any of the embodiments above, wherein the plurality of apertures are disposed in the helical valley

Alternatively or additionally to any of the embodiments above, wherein the plurality of apertures extend along a length of the elongate shaft corresponding to a length of a stent formed in-situ.

Alternatively or additionally to any of the embodiments above, the delivery system, further comprising a stent forming material source.

Alternatively or additionally to any of the embodiments above, wherein the stent forming material source is configured to mate with the port.

Alternatively or additionally to any of the embodiments above, wherein the stent forming material source is a spray can.

Alternatively or additionally to any of the embodiments above, wherein the stent forming material source is a syringe

Alternatively or additionally to any of the embodiments above, further comprising a distal seal positioned within the valleys distal to the plurality of apertures.

Alternatively or additionally to any of the embodiments above, further comprising a proximal seal positioned within the valleys proximal to the plurality of apertures.

Alternatively or additionally to any of the embodiments above, wherein an outer surface of the elongate shaft is lubricious.

An example method for forming a stent in-situ comprises:

advancing a delivery system within a body lumen, the delivery system comprising:
an elongate shaft having a proximal end and a distal end;
a lumen extending from the proximal end towards the distal end of the elongate shaft;
a plurality of apertures adjacent a distal end region of the elongate shaft, the plurality of apertures extending from an outer surface to an inner surface of the elongate shaft and in fluid communication with the lumen; and
a port affixed to the proximal end of the elongate shaft and in fluid communication with the lumen.

positioning the plurality of apertures adjacent to a desired treatment region;

connecting a stent forming material source to the port of the delivery system;

introducing a stent forming material into the lumen of the elongate shaft and out the plurality of apertures into the body lumen;

removing the delivery system from the body lumen; and

allowing the stent forming material to cure.

An example delivery system for forming a stent in-situ comprises:

an elongate shaft having a proximal end and a distal end;

a lumen extending from the proximal end towards the distal end of the elongate shaft;

a plurality of apertures adjacent a distal end region of the elongate shaft, the plurality of apertures extending from an outer surface to an inner surface of the elongate shaft and in fluid communication with the lumen; and

a port affixed to the proximal end of the elongate shaft and in fluid communication with the lumen.

Alternatively or additionally to any of the embodiments above, wherein the outer surface of the elongate shaft comprises a plurality of radially extending ridges and a plurality of valleys extending along a length of the elongate shaft.

Alternatively or additionally to any of the embodiments above, wherein the plurality apertures are disposed within at least one valley of the plurality of valleys.

Alternatively or additionally to any of the embodiments above, wherein the plurality of apertures comprises at least one aperture disposed in each valley of the plurality of valleys.

Alternatively or additionally to any of the embodiments above, wherein the plurality of apertures comprises a two or more apertures disposed in each valley of the plurality of valleys.

Alternatively or additionally to any of the embodiments above, wherein the two or more apertures are arranged in a linear array.

Alternatively or additionally to any of the embodiments above, wherein the plurality of apertures extend along a length of the elongate shaft corresponding to a length of a stent formed in-situ.

Alternatively or additionally to any of the embodiments above, wherein the outer surface of the elongate shaft comprises a radially extending helical ridge and a helical valley extending along a length of the elongate shaft.

Alternatively or additionally to any of the embodiments above, wherein the plurality of apertures are disposed in the helical valley.

Alternatively or additionally to any of the embodiments above, further comprising a stent forming material source.

Alternatively or additionally to any of the embodiments above, wherein the stent forming material source is configured to be mate with the port.

Alternatively or additionally to any of the embodiments above, wherein the stent forming material source is a spray can.

Alternatively or additionally to any of the embodiments above, wherein the stent forming material source is a syringe

Alternatively or additionally to any of the embodiments above, further comprising a distal seal positioned within the valleys distal to the plurality of apertures.

Alternatively or additionally to any of the embodiments above, further comprising a proximal seal positioned within the valleys proximal to the plurality of apertures.

Alternatively or additionally to any of the embodiments above, further comprising a proximal seal positioned within the valleys proximal to the plurality of apertures and a comprising a distal seal positioned within the valleys distal to the plurality of apertures.

Alternatively or additionally to any of the embodiments above, wherein an outer surface of the elongate shaft is lubricious.

An example method for forming a stent in-situ comprises:

advancing a delivery system within a body lumen, the delivery system comprising:
an elongate shaft having a proximal end and a distal end;
a lumen extending from the proximal end towards the distal end of the elongate shaft;
a plurality of apertures adjacent a distal end region of the elongate shaft, the plurality of apertures extending from an outer surface to an inner surface of the elongate shaft and in fluid communication with the lumen; and
a port affixed to the proximal end of the elongate shaft and in fluid communication with the lumen.

positioning the plurality of apertures adjacent to a desired treatment region;

connecting a stent forming material source to the port of the delivery system;

introducing a stent forming material into the lumen of the elongate shaft and out the plurality of apertures into the body lumen;

removing the delivery system from the body lumen; and

curing the stent forming material.

Alternatively or additionally to any of the embodiments above, wherein curing the stent forming material comprises a chemical reaction.

Alternatively or additionally to any of the embodiments above, wherein introducing a stent forming material into the lumen of the elongate shaft comprises actuating an actuation mechanism on the stent forming material source.

Alternatively or additionally to any of the embodiments above, wherein the stent forming material comprises a foam, a two-part epoxy, or a liquid polymer system.

Alternatively or additionally to any of the embodiments above, wherein the outer surface of the elongate shaft comprises at least one radially extending ridge and at least one valley extending along a length of the elongate shaft.

An example method for forming a stent in-situ comprises:

advancing a delivery system within a body lumen, the delivery system comprising:
an elongate tubular shaft having an inner surface, an outer surface, a proximal end and a distal end, the outer surface comprising a plurality of longitudinally extending alternative ridges and valleys;
a lumen extending from the proximal end towards the distal end of the elongate tubular shaft;
a plurality of apertures disposed within at least one of the plurality of valleys and adjacent to a distal end region of the elongate tubular shaft, the plurality of apertures extending from the outer surface to the inner surface of the elongate tubular shaft and in fluid communication with the lumen; and
a port affixed to the proximal end of the elongate tubular shaft and in fluid communication with the lumen.

positioning the plurality of apertures adjacent to a desired treatment region;

connecting a stent forming material source to the port of the delivery system;

introducing a stent forming material into the lumen of the elongate shaft and out the plurality of apertures into the body lumen;

removing the delivery system from the body lumen; and

allowing the stent forming material to cure after removing the delivery system from the body lumen.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 is a perspective view of an illustrative delivery system for delivery of an in-situ formed stent.

FIG. 2 is a close up perspective view of the distal end of the illustrative delivery system of FIG. 1.

FIG. 3 is a cross-section of an esophagus with a stricture.

FIG. 4 is a cross-section of an illustrative delivery system within the esophagus.

FIG. 5 is another cross-section of an illustrative delivery system within the esophagus.

FIG. 6 is a cross-section of the in-situ formed stent within the esophagus.

FIG. 7 is a close up perspective view of a distal end of another illustrative delivery system.

While the disclosure is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency in patients with esophageal strictures. Such stents may be used in patients experiencing dysphagia, sometimes due to esophageal cancer. An esophageal stent may allow a patient to maintain nutrition via oral intake during cancer treatment or palliation periods. In some instances, it may be desirable to form the stent in-situ or within the body lumen. A fluent (fluid or otherwise flowable), pre-stent or stent forming material may be delivered through a delivery system and cured or hardened within the body lumen to form a stent. While the embodiments disclosed herein are discussed with reference to esophageal stents, it is contemplated that the delivery systems and/or stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, biliary tract, urinary tract, prostate, brain, stomach and the like.

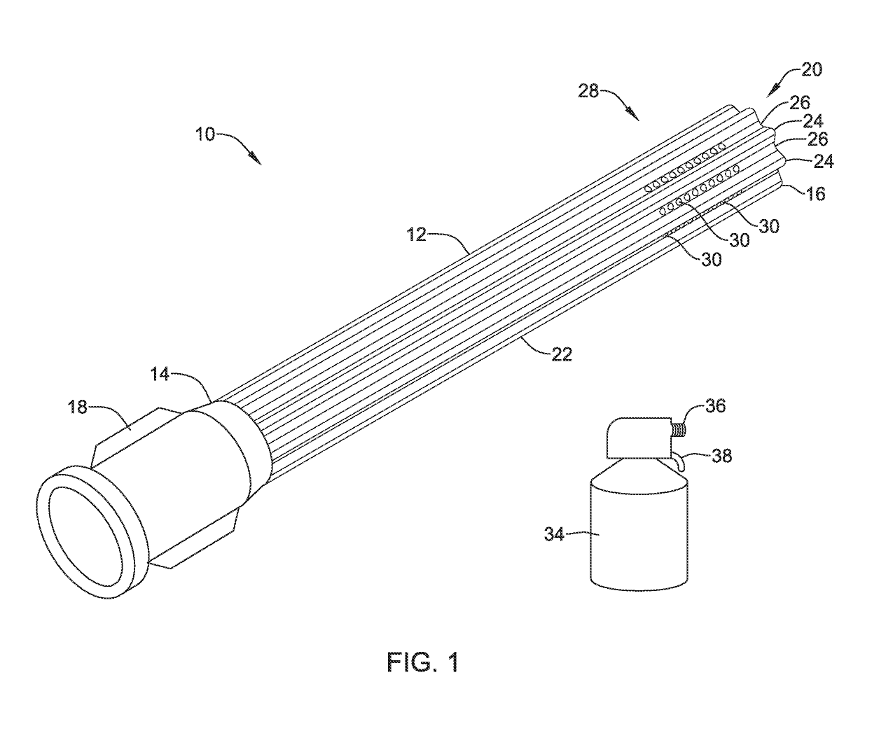

FIG. 1 illustrates a perspective view of an illustrative in-situ stent delivery system 10. The delivery system 10 may be used to deliver a flowable stent forming material to a desired treatment location where it is then cured or hardened to form a stent in-situ. The delivery system 10 may include an elongate tubular shaft 12 having a proximal end 14 and a distal end 16. The proximal end 14 of the elongate shaft 12 may include a port 18, such as, but not limited to, a Luer connection, affixed or attached thereto for connecting other treatment devices or providing a port for facilitating other treatments. The port 18 may be configured to remain outside the body. It is contemplated that the stiffness and size of the elongate shaft 12 may be modified to form a delivery system 10 for use in various locations within the body. The elongate shaft 12 may further include a lumen 20 extending from the proximal end 14 and towards the distal end 16. The lumen 20 may be in fluid communication with the port 18. In some embodiments, the lumen 20 may terminate proximal to the distal end 16 of the elongate shaft 12 such that fluid or other treatments do not exit through the distal end 16 of the elongate shaft 12. In other embodiments, the lumen 20 may extend to a distal opening at the distal end 16 of the elongate shaft 12. While not explicitly shown, the elongate shaft 12 may include one or more additional lumens through which a guidewire (not explicitly shown) may be passed in order to advance the elongate shaft 12 to a predetermined position, although this is not required. The delivery system 10 may be configured to be advanced through a working channel of an endoscope, gastroscope or other guide means.

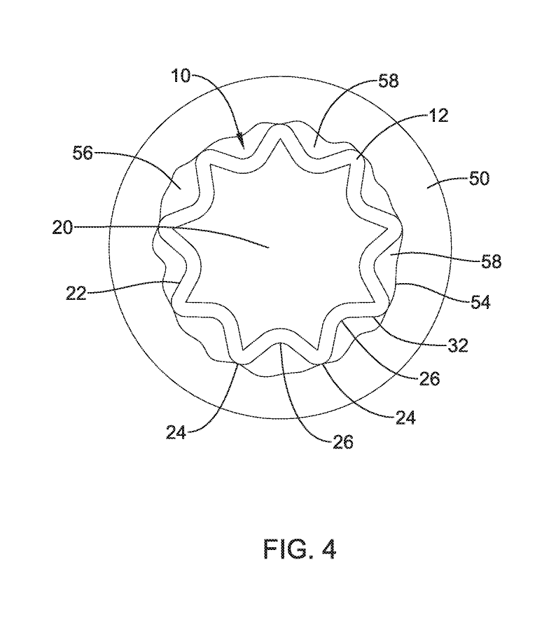

An outer surface 22 of the elongate shaft 12 may include a plurality of alternating radially extending bumps or ridges 24 extending along a length of the elongate shaft 12. A dip or valley 26 may be disposed between adjacent ridges 24 to define a plurality of valleys 26. The undulating ridges 24 and valleys 26 may give the elongate shaft 12 a star-like cross sectional shape, as shown in FIG. 4. It is contemplated that the number of ridges 24 and valleys 26 may be selected based on the desired application. It is further contemplated that the elongate shaft 12 may have other cross-sectional shapes, such as but not limited to, circular, oblong, rectangular, polygonal, etc. The ridges 24 may be sized to apply a radially outward pressure to a stricture to allow a distal end region 28 of the elongate shaft 12 to be positioned adjacent to the stricture. The valleys 26 may create a space between the esophagus and the elongate shaft 12, as will be discussed in more detail below. In some embodiments, the ridges 24 and/or valleys 26 may extend from the proximal end 14 to the distal end 16 of the elongate shaft 12, as shown. In other embodiments, the ridges 24 and/or valleys 26 may extend along a portion of the elongate shaft 12, such as, but not limited to, the distal end region 28. For example, the ridges 24 and/or valleys 26 may extend from a point distal to the proximal end 14 of the elongate shaft 12 to the distal end 16. This is just an example. It is contemplated that the ridges 24 and/or valleys 26 may be positioned at any longitudinal position along the length of the elongate shaft 12 and may extend over any desired length.

While the elongate shaft 12 is illustrated as including a plurality of ridges 24 and valleys 26, it is contemplated that the elongate shaft 12 may include any number of ridges 24 and/or valleys 26 desired. In some embodiments, the elongate shaft 12 may include a single ridge 24 and a single valley 26. It is further contemplated that the number of ridges 24 and valleys 26 may not be the same. The size, shape, and/or configuration of the ridges 24 and/or valleys 26 may be selected to achieve a desired stent shape. The ridges 24 and/or valleys 26 may be in non-linear configurations. For example, the ridges 24 and/or valleys 26 may have linear configuration, a curved or arc shape, a sinusoidal or undulating shape, a helical shape, a partial helical shape, or combinations thereof.

The delivery system 10 may further include a stent forming material source 34 configured to be attached to the port 18. In some embodiments, the stent forming material source may be spray can 34 including a nozzle 36 and a trigger 38 for deploying the material. In some instances, the spray can 34 may be an aerosolizing spray can. It is contemplated that the nozzle 36 be structured to mate with the port 18. For example, if the port 18 is a female Luer connection, the nozzle 36 may be a male Luer connection. The reverse configuration is also contemplated. The stent forming material source is not limited to a spray can. Other sources are also contemplated. For example, the source 34 may be a syringe or a pump system.

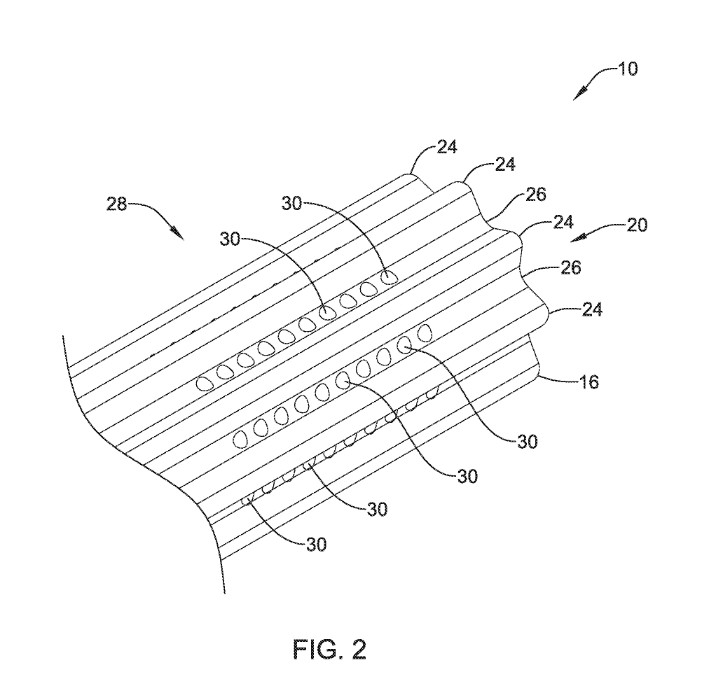

FIG. 2 illustrates a close up perspective view of the distal end region 28 of the illustrative delivery system 10 of FIG. 1. The delivery system 10 may further include a plurality of through holes or apertures 30 extending from the outer surface 22 of the elongate shaft 12 to an inner surface of the elongate shaft 12. The apertures 30 may be in fluid communication with the lumen 20 of the elongate shaft 12. It is contemplated that the apertures 30 may be positioned within each of the valleys 26 to allow for delivery of a stent forming material. The apertures 30 may be spaced along a length of each valley 26. In some instances, the apertures 30 may be spaced along a length of the valleys 26 that is similar in length to a desired length of an in-situ formed stent. In some embodiments, the apertures 30 may be positioned in a linear array within each valley 26, as shown in FIG. 2. In other embodiments, the apertures 30 may be staggered, arranged in multiple rows, or otherwise patterned. In some instances, the pattern of the apertures 30 may generally correspond to the desired final shape of the stent. Further, there may be any number of apertures 30 formed in each valley 26, as desired, to allow a desired amount of stent forming material to be delivered through the lumen 20 and out through the apertures 30 to the desired treatment location. It is further contemplated that the size and spacing of the apertures 30 may be varied to deliver the desired amount of stent forming material to the desired treatment location.

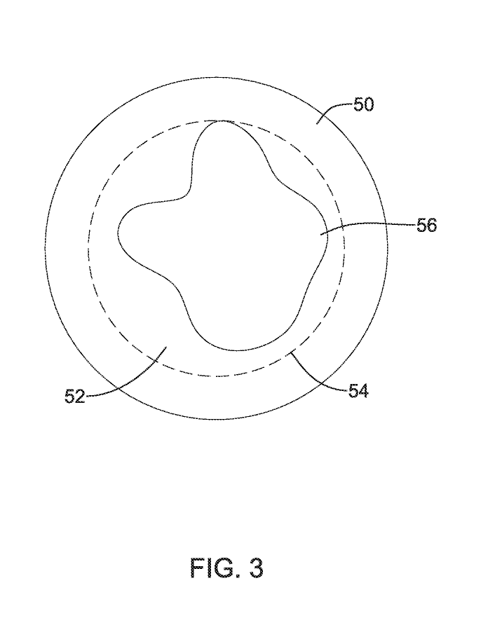

As discussed above, the delivery system 10 may be used to deliver a stent forming material to a desired treatment region to form a stent in-situ. A method for forming a stent in-situ will now be described with respect to FIGS. 3-6. FIG. 3 illustrates a cross-sectional view of an esophageal wall 50 with a stricture 52. The stricture 52 may reduce the cross-sectional area 56 of the esophagus. Dashed line 54 may represent an average or desired cross-section of the esophagus. To deliver the stent forming material, the delivery system 10 may be advanced down the esophagus until the distal end region 28 is adjacent the stricture 52. It is contemplated that the delivery system 10 may be advanced with or without the use of an endoscope or other guide means. As shown in FIG. 4, which illustrates a cross-section of the delivery system 10 within the esophagus, the elongate shaft 12 of the delivery system 10 may be sized such that it pushes against the stricture and opens the esophagus. The ridges 24 of the elongate shaft 12 may push against the esophageal wall 50 to open it to a dimension similar to an average or desired cross-section 54. The valleys 26 of the elongate shaft 12 may create a plurality of spaces or openings 58 between the esophageal wall 50 and the elongate shaft 12.

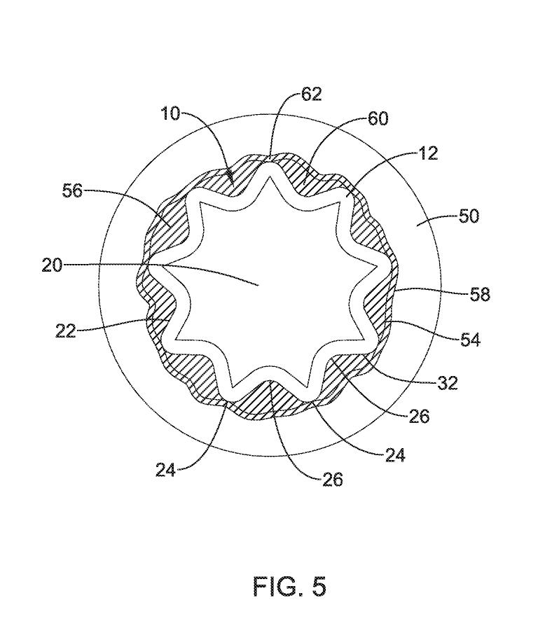

Once the delivery system 10 is positioned with the distal end region 28 and the apertures 30 adjacent to the treatment location, a stent forming material source 34 may be connected to the port 18 of the delivery system 10. It is contemplated that the delivery of the stent forming material may be controlled through actuation of an actuation mechanism on the stent forming material source 34, such as, but not limited to a trigger, handle, plunger, etc. The stent forming material may be a foam spray, a two part epoxy, a liquid polymer system, or otherwise flowable, biocompatible material. The stent forming material 60 may be introduced into the lumen 20 of the delivery system 10 through the port 18 as an uncured material. The stent forming material 60 may flow through the lumen 20 and exit the delivery system 10 through the apertures 30 adjacent the distal end region 28, as shown in FIG. 5. As discussed above, the lumen 20 may terminate or have an end wall (not explicitly shown) proximal to the distal end 16 of the elongate shaft 12 to prevent the stent forming material 60 from flowing out of the distal end 16 of the delivery system 10.

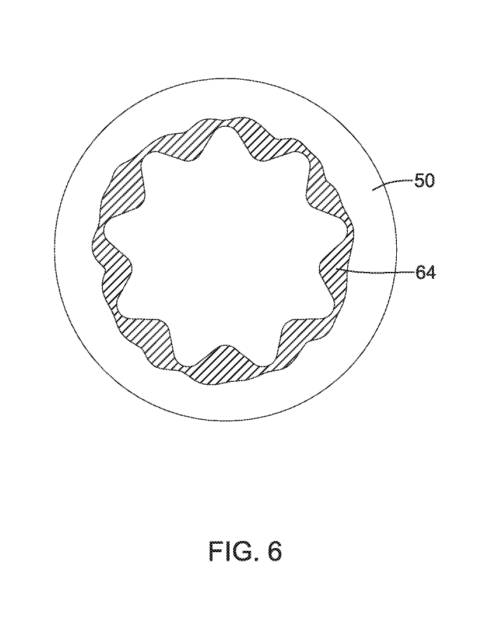

The stent forming material 60 may cross-link, cure, harden, or otherwise change from a flowable material into a rigid stent 64 (see FIG. 6) capable of applying a radially outward pressure to the stricture 52 in the esophagus to open the lumen and allow for the passage of foods, fluids, air, etc. In some embodiments, the phase change from liquid to solid may be caused by a chemical reaction to the moisture in the esophagus or by a chemical reaction with another component delivered simultaneously with the stent forming material 60. It is contemplated that no external energy source is necessary to cause the stent forming material 60 to harden. The stent forming material 60 may be delivered into the openings 58 between the elongate shaft 12 and the esophageal wall 50.

As the stent forming material 60 polymerizes or cures, the material may expand such that the esophageal wall 50 is further compressed to further enlarge the cross-sectional area 56 of the esophagus, as shown at reference number 58. The expansion of the stent forming material 60 may also allow the stent forming material 60 to flow or extend into the region 62 between the ridges 24 and the esophageal wall 50. This may allow the stent forming material 60 to form a continuous or solid, generally tubular, stent 64 within the esophagus, as shown in FIG. 6. The stent forming material 60 may polymerize or cure relatively quickly once it has been delivered into the esophagus. This may help prevent undesirable migration of the stent forming material 60. As the material 60 cures in close proximity to where it exits the elongate shaft 12, the length of the stent 64 may generally correspond to the length of the elongate shaft 12 over which the apertures 30 extend. It is further contemplated that the stent 64 may have an undulating inner surface generally corresponding to the shape of the outer surface 22 of the elongate shaft 12. The outer surface of the stent 64 may generally conform to the shape of the esophagus.

The delivery system 10 may be removed from the esophagus prior to the stent forming material 60 being fully cured or hardened. The stent forming material 60 may continue to cure or harden after the delivery system 10 has been removed. In other embodiments, the delivery system 10 may be removed from the esophagus once the stent forming material 60 has polymerized or cured. In some embodiments, the outer surface 22 of the elongate shaft 12 may have a non-stick or lubricious coating 32 to facilitate removal of the delivery system 10. In other embodiments, the elongate shaft 12 may be formed from a lubricious material. Lubricious materials and/or coatings may include, but not limited to, polytetrafluoroethylene (PTFE), polyethylene (PE), polypropylene (PP), polyvinylchloride (PVC), ethylvinylacetate (EVA), polyurethanes, polyamides, polyethyleneteraphthalate (PET), ethylene-chlorofluoroethylene (ECTFE), fluorinated ethylenepropylene (FEP), polychlorotrifluoroethylene (PCTFE), polyvinylfluoride (PVF), polyvinylidenefluoride (PVDF), and their mixtures and/or copolymers thereof. These are just examples. It is contemplated that the elongate shaft 12 and/or coating 32 may be formed of any material that reduces friction between the foaming material 60 and the elongate shaft 12.

In some embodiments, the delivery system 10 may not include, or may be free from, a proximal and/or distal seal to define a fill region (e.g. the region/length over which the stent 64 is intended to extend). Instead, the delivery system 10 may rely on the viscosity and/or a quick curing time of the stent forming material 60 to prevent the stent forming material 60 from migrating the undesired areas. In other embodiments, a proximal and/or distal seal may be provided on the outer surface 22 of the elongate shaft 12 to define a fill region. The proximal and/or distal seal may fill the valleys 26 at a longitudinal point along the elongate shaft 12 to prevent migration of the stent forming material 60. For example, a proximal seal may be provided at a location proximal to the proximal-most aperture 30 and a distal seal may be provided at a location distal to the distal-most aperture 30. It is contemplated that a distal seal (and/or proximal seal) may be configured to be collapsible, crushable, or otherwise deformable to facilitate removal of the delivery system 10 from the esophagus after the stent 64 has been formed.

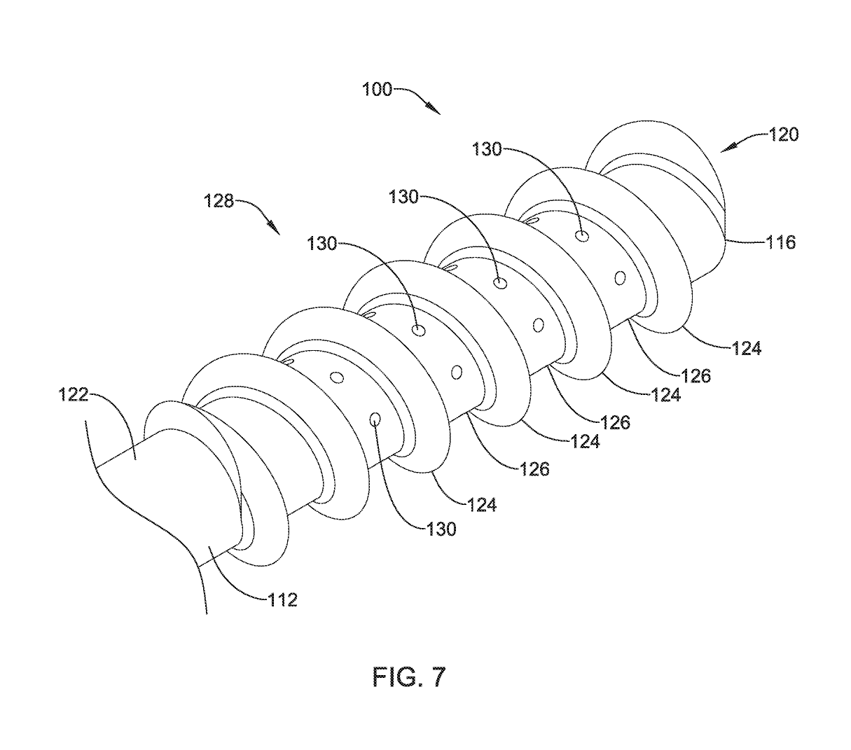

FIG. 7 illustrates a close up perspective view of the distal end region 128 of another illustrative delivery system 100. The delivery system 100 may be used to deliver a flowable stent forming material to a desired treatment location where it is then cured or hardened to form a stent in-situ. The delivery system 100 may be similar in form and function to the delivery system 10 described above. The delivery system 100 may include an elongate tubular shaft 112 having a proximal end (not explicitly shown) and a distal end 116. While not explicitly shown, the proximal end of the elongate shaft 112 may include a port, such as, but not limited to, a Luer connection, affixed or attached thereto for connecting other treatment devices or providing a port for facilitating other treatments. The port may be configured to remain outside the body. It is contemplated that the stiffness and size of the elongate shaft 112 may be modified to form a delivery system 100 for use in various locations within the body. The elongate shaft 112 may further include a lumen 120 extending from the proximal end and towards the distal end 116. The lumen 120 may be in fluid communication with the port. In some embodiments, the lumen 120 may terminate proximal to the distal end 116 of the elongate shaft 112 such that fluid or other treatments do not exit through the distal end 116 of the elongate shaft 112. In other embodiments, the lumen 120 may extend to a distal opening at the distal end 116 of the elongate shaft 112. While not explicitly shown, the elongate shaft 112 may include one or more additional lumens through which a guidewire (not explicitly shown) may be passed in order to advance the elongate shaft 112 to a predetermined position, although this is not required. The delivery system 100 may be configured to be advanced through a working channel of an endoscope, gastroscope or other guide means. The delivery system 100 may further include a stent forming material source, such as the stent forming material source 34 described above, configured to be attached to the port.

An outer surface 122 of the elongate shaft 112 may include a radially extending helical bump or ridge 124 and a helical dip or valley 126 extending along a length of the elongate shaft 112. The ridge 124 may be one continuous ridge or a plurality of interconnected ridges. The valley 126 may be a continuous valley extending about a perimeter of the elongate shaft 112 in a helical manner. The ridge 124 may be sized and shaped to apply a radially outward pressure to a stricture to allow a distal end region 128 of the elongate shaft 112 to be positioned adjacent to the stricture. The valley 126 may create a space between the esophagus and the elongate shaft 112. In some embodiments, the ridge 124 and/or valley 126 may extend from the proximal end to the distal end 116 of the elongate shaft 112. In other embodiments, the ridge 124 and/or valley 126 may extend along a portion of the elongate shaft 112, such as, but not limited to, the distal end region 128. For example, the ridge 124 and/or valley 126 may extend from a point distal to the proximal end of the elongate shaft 112 to the distal end 116. This is just an example. It is contemplated that the ridge 124 and/or valley 126 may be positioned at any longitudinal position along the length of the elongate shaft 112 and may extend over any desired length.

The delivery system 100 may further include a plurality of through holes or apertures 130 extending from the outer surface 122 of the elongate shaft 112 to an inner surface of the elongate shaft 112. The apertures 130 may be in fluid communication with the lumen 120 of the elongate shaft 112. It is contemplated that the apertures 130 may be positioned within the valley 126 to allow for delivery of a stent forming material. The apertures 130 may be spaced along the valley 126 to distribute a stent forming material radially and longitudinally from the elongate shaft 112. In some instances, the apertures 130 may be spaced along a length of the elongate shaft 112 and/or valley 126 that is similar in length to a desired length of an in-situ formed stent. In some embodiments, the apertures 130 may be positioned in an array within the valley 126. In other embodiments, the apertures 130 may be staggered, arranged in multiple rows, or otherwise patterned. In some instances, the pattern of the apertures 130 may generally correspond to the desired final shape of the stent. Further, there may be any number of apertures 130 formed in the valley 126, as desired, to allow a desired amount of stent forming material to be delivered through the lumen 120 and out through the apertures 130 to the desired treatment location. It is further contemplated that the size and spacing of the apertures 130 may be varied to deliver the desired amount of stent forming material to the desired treatment location.

While not explicitly shown, the delivery system 100 may be used to deliver a stent forming material to a desired treatment region to form a stent in-situ in a similar manner to the method described with respect FIGS. 3-6. To deliver the stent forming material, the delivery system 100 may be advanced down the esophagus until the distal end region 128 is adjacent the stricture. It is contemplated that the delivery system 100 may be advanced with or without the use of an endoscope or other guide means. The elongate shaft 112 of the delivery system 100 may be sized such that it pushes against the stricture and opens the esophagus. The ridge 124 of the elongate shaft 112 may push against the esophageal wall to open it to a dimension similar to an average or desired cross-section. The valley 126 of the elongate shaft 112 may create a continuous, helical space between the esophageal wall and the elongate shaft 112.

Once the delivery system 100 is positioned with the distal end region 128 and the apertures 130 adjacent to the treatment location, a stent forming material source may be connected to the port of the delivery system 100. It is contemplated that the delivery of the stent forming material may be controlled through actuation of an actuation mechanism on the stent forming material source, such as, but not limited to a trigger, handle, plunger, etc. The stent forming material may be a foam spray, a two part epoxy, a liquid polymer system, or otherwise flowable, biocompatible material. The stent forming material may be introduced into the lumen 120 of the delivery system 100 through the port as an uncured material. The stent forming material may flow through the lumen 120 and exit the delivery system 100 through the apertures 130 adjacent the distal end region 128. As discussed above, the lumen 120 may terminate or have an end wall (not explicitly shown) proximal to the distal end 116 of the elongate shaft 112 to prevent the stent forming material from flowing out of the distal end 116 of the delivery system 100.

The stent forming material may cross-link, cure, harden, or otherwise change from a flowable material into a rigid stent capable of applying a radially outward pressure to the stricture in the esophagus to open the lumen and allow for the passage of foods, fluids, air, etc. In some embodiments, the phase change from liquid to solid may be caused by a chemical reaction to the moisture in the esophagus or by a chemical reaction with another component delivered simultaneously with the stent forming material. It is contemplated that no external energy source is necessary to cause the stent forming material to harden. The stent forming material may be delivered into the openings between the elongate shaft and the esophageal wall. As the stent forming material polymerizes or cures, the material may expand such that the esophageal wall is further compressed to further enlarge the cross-sectional area of the esophagus. The expansion of the stent forming material may also allow the stent forming material to flow or extend into the region between the ridge 124 and the esophageal wall. This may allow the stent forming material to form a continuous or solid, generally tubular, stent within the esophagus, although this is not required. It is contemplated that the ridge 124 may be sized and/or shaped such that the expansion of the stent forming material does not allow the material from adjacent windings of the valley 126 to connect. This may result in a helical, or spring-shaped, stent.

As discussed above, the size, shape, and/or configuration of the ridge 124 and/or valley 126 may be selected to achieve any final stent shape desired. In some instances, the stent may have a generally tubular shape. In other instances, the stent may have a helical shape. It is further contemplated that the final stent shape may have a plurality of interconnected struts forming a variety of patterns defining a plurality of open, cellular regions.

The stent forming material may polymerize or cure relatively quickly once it has been delivered into the esophagus. This may help prevent undesirable migration of the stent forming material. As the material cures in close proximity to where it exits the elongate shaft 112, the length of the stent may generally correspond to the length of the elongate shaft 112 over which the apertures 130 extend. It is further contemplated that the stent may have an inner surface generally corresponding to the shape of the outer surface 122 of the elongate shaft 112. The outer surface of the stent may generally conform to the shape of the esophagus.

The delivery system 100 may be removed from the esophagus prior to the stent forming material being fully cured or hardened. The stent forming material may continue to cure or harden after the delivery system 100 has been removed. In other embodiments, the delivery system 100 may be removed from the esophagus once the stent forming material has polymerized or cured. In some embodiments, the outer surface 122 of the elongate shaft 112 may have a non-stick or lubricious coating to facilitate removal of the delivery system 100. In other embodiments, the elongate shaft 112 may be formed from a lubricious material.

In some embodiments, the delivery system 100 may not include, or may be free from, a proximal and/or distal seal to define a fill region (e.g. the region/length over which the stent is intended to extend). Instead, the delivery system 100 may rely on the viscosity and/or a quick curing time of the stent forming material to prevent the stent forming material from migrating the undesired areas. In other embodiments, a proximal and/or distal seal may be provided on the outer surface 122 of the elongate shaft 112 to define a fill region. The proximal and/or distal seal may fill the valley 126 at a longitudinal point along the elongate shaft 112 to prevent migration of the stent forming material. For example, a proximal seal may be provided at a location proximal to the proximal-most aperture 130 and a distal seal may be provided at a location distal to the distal-most aperture 130. It is contemplated that a distal seal (and/or proximal seal) may be configured to be collapsible, crushable, or otherwise deformable to facilitate removal of the delivery system 10 from the esophagus after the stent has been formed.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. In some other embodiments, a superelastic alloy, for example a superelastic nitinol can be used to achieve desired properties.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

In some embodiments, a degree of Magnetic Resonance Imaging (MM) compatibility is imparted into the stents or delivery systems. For example, the stents or delivery systems or portions thereof, may be made of a material that does not substantially distort the image and create substantial artifacts (i.e., gaps in the image). Certain ferromagnetic materials, for example, may not be suitable because they may create artifacts in an MRI image. The stents or delivery systems or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments.

What is claimed is:

1. A delivery system for forming a stent in-situ, the system comprising:
an elongate shaft having a proximal end and a distal end;
a lumen extending from the proximal end towards the distal end of the elongate shaft;
a plurality of apertures adjacent a distal end region of the elongate shaft, the plurality of apertures extending from an outer surface to an inner surface of the elongate shaft and in fluid communication with the lumen; and
a port affixed to the proximal end of the elongate shaft and in fluid communication with the lumen.

2. The delivery system of claim 1, wherein the outer surface of the elongate shaft comprises a plurality of radially extending ridges and a plurality of valleys extending along a length of the elongate shaft.

3. The delivery system of claim 2, wherein the plurality apertures are disposed within at least one valley of the plurality of valleys.

4. The delivery system of claim 2, wherein the plurality of apertures comprises at least one aperture disposed in each valley of the plurality of valleys.

5. The delivery system of claim 2, wherein the plurality of apertures comprises a two or more apertures disposed in each valley of the plurality of valleys.

6. The delivery system of claim 5, wherein the two or more apertures are arranged in a linear array.

7. The delivery system of claim 1, wherein the plurality of apertures extend along a length of the elongate shaft corresponding to a length of a stent formed in-situ.

8. The delivery system of claim 1, wherein the outer surface of the elongate shaft comprises a radially extending helical ridge and a helical valley extending along a length of the elongate shaft.

9. The delivery system of claim 8, wherein the plurality of apertures are disposed in the helical valley.

10. The delivery system of claim 1, further comprising a stent forming material source.

11. The delivery system of claim 10, wherein the stent forming material source is configured to mate with the port.

12. The delivery system of claim 10, wherein the stent forming material source is a spray can.

13. The delivery system of claim 10, wherein the stent forming material source is a syringe

14. The delivery system of claim 1, wherein an outer surface of the elongate shaft is lubricious.

15. A method for forming a stent in-situ, the method comprising:
advancing a delivery system within a body lumen, the delivery system comprising:
an elongate shaft having a proximal end and a distal end;
a lumen extending from the proximal end towards the distal end of the elongate shaft;
a plurality of apertures adjacent a distal end region of the elongate shaft, the plurality of apertures extending from an outer surface to an inner surface of the elongate shaft and in fluid communication with the lumen; and
a port affixed to the proximal end of the elongate shaft and in fluid communication with the lumen.
positioning the plurality of apertures adjacent to a desired treatment region;
connecting a stent forming material source to the port of the delivery system;
introducing a stent forming material into the lumen of the elongate shaft and out the plurality of apertures into the body lumen;
removing the delivery system from the body lumen; and
curing the stent forming material.

16. The method of claim 15, wherein curing the stent forming material comprises a chemical reaction.

17. The method of claim 15, wherein introducing a stent forming material into the lumen of the elongate shaft comprises actuating an actuation mechanism on the stent forming material source.

18. The method of claim 15, wherein the stent forming material comprises a foam, a two-part epoxy, or a liquid polymer system.

19. The method of claim 15, wherein the outer surface of the elongate shaft comprises at least one radially extending ridge and at least one valley extending along a length of the elongate shaft.

20. A method for forming a stent in-situ, the method comprising:
advancing a delivery system within a body lumen, the delivery system comprising:
an elongate tubular shaft having an inner surface, an outer surface, a proximal end and a distal end, the outer surface comprising a plurality of longitudinally extending alternative ridges and valleys;
a lumen extending from the proximal end towards the distal end of the elongate tubular shaft;
a plurality of apertures disposed within at least one of the plurality of valleys and adjacent to a distal end region of the elongate tubular shaft, the plurality of apertures extending from the outer surface to the inner surface of the elongate tubular shaft and in fluid communication with the lumen; and
a port affixed to the proximal end of the elongate tubular shaft and in fluid communication with the lumen.
positioning the plurality of apertures adjacent to a desired treatment region;
connecting a stent forming material source to the port of the delivery system;
introducing a stent forming material into the lumen of the elongate shaft and out the plurality of apertures into the body lumen;
removing the delivery system from the body lumen; and
allowing the stent forming material to cure after removing the delivery system from the body lumen.
